(19) United States
(12) Patent Application Publication
(10) Pub. No.: US 2003/0003135 A1
(43) Pub. Date: Jan. 2, 2003

(54) Article for drug delivery and methods of making and using same

(76) Inventors: Jeffrey C. Leung, Raleigh, NC (US); Farshid Guilak, Durham, NC (US); Franklin T. Moutos, Durham, NC (US); Anthony V. Seaber, Hillsborough, NC (US)

Correspondence Address: JENKINS & WILSON, PA, 3100 TOWER BLVD, SUITE 1400, DURHAM, NC, 27707, US

(21) Appl. No.: 09/894,067

(22) Filed: Jun. 28, 2001

(51) Int. Cl. A61K 9/70; B32B 27/12

(52) U.S. Cl. 424/443; 442/123

(57) ABSTRACT

A drug carrier, and methods of making and using the same. The drug carrier includes a scaffold including a plurality of layers and a fiber system interconnecting the plurality of layers, wherein the layers define internal and superficial positions for the scaffold; and a drug releasably engaged with the scaffold according to a controlled release profile, wherein the drug is releasably engaged at at least one of the internal or superficial positions. Optionally, the scaffold is a three-dimensional fiber scaffold including at least three systems of fibers, wherein two of the three fiber systems define the plurality of layers, wherein one of the at least three fiber systems interconnects the plurality of layers, and wherein the three dimensions of the scaffold define internal and superficial positions within the scaffold. The drug carrier design is versatile, such that drug release profiles can be customized for a given application.

### TECHNICAL FIELD

The present invention relates to a carrier article constructed for drug delivery. In a preferred embodiment, the carrier comprises a three-dimensional fiber scaffold. The carrier is characterized by predictable physical characteristics and controlled transfer of one or more drugs to a subject. These features can be customized for an intended drug delivery application.

### BACKGROUND ART

Controlled release systems allow for the provision of drugs in a discrete, predetermined manner. Desirable drug release profiles include acute release, continuous release, and intermittent or cyclic release. Representative benefits of a precise drug release system are optimization of drug dose and simplification of administration regime.

An ideal drug delivery system should comprise inert and biocompatible materials, demonstrate predictable mechanical integrity, accommodate appropiate drug loading, safeguard against accidental drug release, and be versatile for custom drug release. Regarding production and use, an ideal drug delivery system should be fabricated and sterilized by simple methods; administered easily, and if required removed easily; and afford comfort to a patient during use.

A number of drug delivery systems have been developed in the art, and these systems are primarily distinguished based on the mechanism and kinetics of drug release. Currently available technologies for regulating drug release include the following:

A. Oil droplet. A drug is partitioned between oily and aqueous phases of an oily droplet. The drug is released as the oily dispersion approaches equilibrium with the aqueous phase.

B. Encapsulation. A drug is coated with a non-reactive hydrophilic or lipophilic layer. Drug release rate is governed by porosity or dissolution of the coating.

C. Matrix system. A drug is dispersed in particulate form within a hydrophilic or lipophilic polymer. Drug release occurs by leaching of the drug from the matrix, influx of solvent into the matrix, and diffusion from pores or voids within the matrix.

D. Membrane controlled systems. A drug is held in a reservoir bounded by a synthetic homogenous or polymeric membrane. Drug release is regulated by the properties of the membrane.

E. Hollow Fibers. A drug is loaded into a hollow fiber. Drug release takes place by diffusion of the drug from the open ends of the fiber and optionally through pores within the fiber.

F. Activation controlled systems. In these systems, drug release is triggered by a specific environmental stimulus.

The aforementioned drug delivery systems are additionally categorized according to temporal features of drug administration, including immediate acute release, acute release following a delay, and sustained release that is constant during the pendency of the device. Acute release is characterized by a single bolus administration. Sustained release reaches a stable value for the lifetime of the device.

A common limitation among existing drug delivery systems and approaches is a lack of versatility wherein a drug carrier can be alternatively constructed to accomplish multiple drug release profiles. Moreover, an article or device that permits cyclic drug release is not currently disclosed in the art.

The present invention pertains to a carrier for drugs having a unique architecture and physical and drug delivery features that are variably designed for a broad spectrum of applications. The present invention thus provides unprecedented cyclic drug release profiles and simultaneous custom release of multiple drugs from a single carrier. Preferred embodiments of a drug carrier of the present invention are useful as transdermal or implantable drug delivery systems. The drug carrier of the present invention thus meets a long-felt need for such a product, for a method of making the product, and for the method of drug administration using the product.

### SUMMARY OF THE INVENTION

The present invention provides a drug carrier, a method for making the carrier, and a method for using the carrier for drug delivery. The carrier comprises a scaffold comprising a plurality of layers and a fiber system interconnecting the plurality of layers, wherein the layers define internal and superficial positions of the scaffold; and a drug releasably engaged with the scaffold according to a controlled release profile, wherein the drug is releasably engaged at at least one of the internal or superficial positions. Optionally, the scaffold is a three-dimensional fiber scaffold including at least three systems of fibers, wherein two of the three fiber systems define the plurality of layers, wherein one of the at least three fiber systems interconnects the plurality of layers, and wherein the three dimensions of the scaffold define internal and superficial positions within the scaffold.

A method for producing a drug carrier article is also disclosed. The method comprises forming a scaffold comprising a plurality of layers and a fiber system interconnecting the plurality of layers, wherein the layers define internal and superficial positions of the scaffold. Further, a drug is releasably engaged at at least one of the internal or superficial positions so that the drug disengages from the scaffold according to a controlled release profile.

A method for delivering a drug to a subject using a drug carrier is also disclosed. The method comprises providing a drug carrier as disclosed herein above; and placing the carrier at a site of desired exposure to the drug, thereby delivering the drug from the carrier to the subject.

Accordingly, it is an object of the present invention to provide a novel drug carrier. This object is achieved in whole or in part by the present invention.

An object of the invention herein above, having been stated, other objects and aspects of the invention will become evident as presented in the detailed description set forth below.

### BRIEF DESCRIPTION OF THE DRAWINGS

FIG. 1 is a perspective view of an embodiment of a three-dimensional orthogonally woven fiber scaffold product of the present invention;

FIG. 2 is a top view of an embodiment of a three-dimensional orthogonally woven fiber scaffold product of the present invention;

FIG. 3 is a side view of an embodiment of a three-dimensional orthogonally woven fiber scaffold product of the present invention;

FIG. 4 is a side cross-sectional view taken along the line 4-4 in FIG. 1 of an embodiment of a three-dimensional orthogonally woven fiber scaffold product of the present invention;

FIG. 5 is a schematic view of a process used in the manufacture of a three-dimensional fiber scaffold product of the present invention; and

FIGS. 6A-6F are schematic graphical representations of exemplary drug releases profiles provided in accordance with the present invention, wherein dosage is represented on the y-axis, and time is represented on the x-axis.

### DETAILED DESCRIPTION OF THE INVENTION

The present invention provides a drug carrier, a method for making the carrier, and a method for using the carrier for drug delivery. The drug carrier comprises a scaffold comprising a plurality of layers and a fiber system interconnecting the plurality of layers, wherein the layers define internal and superficial positions for the scaffold; and a drug releasably engaged with the scaffold according to a controlled release profile, wherein the drug is releasably engaged at at least one of the internal or superficial positions. The disengagement or release of the drug results in transfer of the drug from the carrier to a subject. Optionally, the scaffold is a three-dimensional fiber scaffold comprising at least three systems of fibers, wherein two of the three fiber systems define the plurality of layers, wherein one of the at least three fiber systems interconnects the plurality of layers, and wherein the three dimensions of the scaffold define internal and superficial positions within the scaffold. The drug carrier design is versatile, such that drug release profiles can be customized for a given application.

The carrier can further comprise a three-dimensional fiber scaffold of at least three systems of fibers, wherein two of the three fiber systems define an upper layer, a lower layer, and a medial layer between the upper layer and the lower layer within the three-dimensional fiber scaffold, wherein one of the at least three fiber systems interconnects the upper layer, the lower layer, and the medial layer, and wherein the three dimensions of the fiber scaffold defined internal and superficial positions within the scaffold; and a drug releasably engaged with the scaffold according to a controlled release profile.

An aspect of the present invention pertains to the versatility of drug carrier design, particularly as it affects controlled drug release. The kinetic profile of drug release is designed during construction of the drug carrier by selection of appropriate fiber composition, geometry, and assembly; by consideration of the biochemical properties of the drug, drug formulation, and method for engagement of the drug with the carrier; and by contemplation of the environment for eventual placement of the drug carrier with regard to its affect on disengagement of the drug from the carrier.

While the following terms are believed to be well understood by one of ordinary skill in the art, the following definitions are set forth to facilitate explanation of the invention.

The term “drug carrier”, as used herein, refers to an article or device that effects transference of a drug from the device to a subject.

The term “drug” is any substance that labels or affects the physical or mental functions of a living organism. A drug can be a chemical compound, a protein, a peptide, a nucleic acid, an imaging agent or any other bioactive agent.

The term “imaging agent” is meant to refer to compounds which can be detected.

The term “controlled release profile”, as used herein, is meant to refer to administration of a drug in a pre-selected manner, wherein the temporal features and dose of drug release are predictable.

The term “acute release”, as used herein, refers to a single bolus administration of a drug.

The term “continuous release”, as used herein, is drug release over a prolonged temporal period. A “continuously increasing release” is encompassed by the term “continuous release”, and refers to a increase of drug release over a prolonged temporal period. The increase can be a linear increase, a step-wise increase, a sharp increase followed by a gradual increase, or any other desirable increase profile. A “continuously decreasing release” is encompassed by the term “continuous release”, and refers to a decrease of drug release over a prolonged temporal period. The decrease can be a linear decrease, a step-wise decrease, a sharp decrease followed by a gradual decrease, or any other desirable decrease profile.

The term “delayed release”, as used herein, is drug release that is delayed for a temporal period of time after the provision of a drug carrier of the present invention to a subject.

The term “intermittent release”, as used herein, is two or more temporal periods of a first amount of drug release, wherein the first amount can be the same or different, interluded by a temporal period of a second, different amount of drug release. For example, the term “intermittent release” can refer to two or more temporal periods of drug release interluded by a temporal period substantially without drug release. Alternatively, the term “intermittent release” can refer to a first temporal period of a first amount of drug release, followed by a second temporal period with a second, different amount of drug release, followed by a third temporal period with still another third, different amount of drug release. The term “intermittent release” can also be used synonymously with “cyclic release” or “non-constant release”.

The present invention provides for a drug carrier for the delivery of a drug or other active agent to a subject suffering from a disease, injury or other medical condition in which the delivery of the drug or other active agent is desirable. In th case of the delivery of a drug to an injury, the term “injury” is meant to refer any injury in a subject amenable to treatment via delivery a drug through the implantation of a drug carrier of the present invention; and can comprise an internal injury. Representative internal injuries can include but are not limited to abdominal trauma, visceral injuries, thoracoabdominal trauma, retroperitoneal organ injury, intraperitoneal injury, intra-abdominal bleeding due to blunt trauma, traumatic and non-traumatic perforation of hollow viscera, penetrating injury, abdominal gunshot injury, abdominal shrapnel, abdominal carcinoma, diaphragmatic rupture, stomal hernia, incisional hernia, inguinal hernia, and umbilical hernia. The term “injury” can thus include small wound and in a preferred embodiment refers to very large wounds (e.g. ≧10 cm×10 cm). Other injuries that can be treated via implantation of a drug carrier of the present invention would be apparent to one of ordinary skill in the art after review of the disclosure herein.

The term “subject”, as used herein, refers to any proposed recipient of the drug engaged with the drug carrier.

### A. Three-Dimensional Fiber Scaffold

Three-dimensional fiber scaffolds having a novel architecture characterized by improved strength, porosity, flexibility, and shrinkage resistance properties are provided in accordance with the present invention. The present invention also provides the engagement of a drug with such a fiber scaffold, whereby disengagement of the drug from the scaffold occurs in a predictable and pre-selected manner.

The preferred at least three fiber systems of the fiber scaffold preferably each comprise a bio-compatible material. Optionally, the bio-compatible material comprises a material selected from the group including, but not limited to, an absorbable material, a non-absorbable material, and combinations thereof.

For drug administration at an external body surface, a drug carrier comprising a non-absorbable material is preferred. Representative non-absorbable materials include but are not limited to polypropylene, polyester, polytetrafluoroethylene (PTFE), expanded PTFE (ePTFE), polyethylene, polyurethane, polyamide, nylon, polyetheretherketone (PEEK), polysulfone, a cellulosic, fiberglass, an acrylic, tantalum, polyvinyl alcohol, carbon, ceramic, a metal, any other medically acceptable non-absorbable fiber, and any combination of the foregoing listed materials.

For drug administration at an internal body site, a drug carrier comprising an absorbable material is preferred. Absorption of the drug carrier provides controlled drug release and obviates the necessity of later removal of a drug carrier that has been implanted or injected. Representative absorbable materials include but are not limited to polyglycolic acid (PGA), polylactic acid (PLA), polyglycolide-lactide, polycaprolactone, polydioxanone, polyoxalate, a polyanhydride, a poly(phosphoester), catgut suture, collagen, silk, chitin, chitosan, hydroxyapatite, bioabsorbable calcium phosphate, hyaluronic acid, any other medically acceptable absorbable fiber, and combinations thereof.

Optionally, the fiber scaffold can comprise both absorbable and non-absorbable fiber systems. In one embodiment, the fiber systems that define the upper layer of the fiber scaffold comprise primarily an absorbable material, the fiber systems that define the lower layer comprise primarily a non-absorbable material, and the fiber systems that define the medial layer comprise both an absorbable material and a non-absorbable material.

In a drug carrier of the present invention, the fiber systems of the fiber scaffold comprise a monofilament fiber, a multifilament fiber, a hollow fiber, a fiber having a variable cross-section along its length, or combinations thereof. In the case of a hollow fiber, at least one end of the fiber is preferably open. Fibers can be any shape, e.g. bracket-shaped (i.e. [), polygonal, square, I-beam star-shaped, T-shaped, clover shaped, “dog bone” shaped, or any other suitable shape. Preferably, the shape and dimensions of the fiber are selected to optimize spatial and temporal features of controlled drug release.

A T-shaped hollow fiber can be constructed by connecting two hollow linear fibers perpendicular to one another, so that an end of one linear fiber is joined to a medial position of the other linear fiber. Preferably, both ends of each constituent linear fiber are open, and the connection of the two linear fibers establishes continuity of the hollow interior of each linear fiber. A T-shaped fiber so constructed has three open ends. A drug loaded within a T-shaped hollow fiber releases the drug at a greater rate than a linear fiber of the same internal diameter having only two open ends (e.g., one of the constituent linear fibers of the T-shaped hollow fiber).

The dimensions of a fiber can also be selected to regulate a rate of drug release. For example, an open-ended hollow fiber with a relatively large internal diameter will release a loaded drug at a greater rate than an identically-shaped open-ended hollow fiber with a smaller internal diameter.

The shape of fibers can also dictate the sites of drug release. In the case of a hollow fiber, the sites of drug release correspond to the positions of the open ends of the fiber. Thus, a T-shaped hollow fiber, described herein above, releases a loaded drug at three sites, corresponding to the positions of its three open ends. By contrast, a linear hollow fiber will release a loaded drug at two sites, corresponding to the positions of its two open ends.

Optionally, the fiber scaffold comprises a plurality of contact points among the at least three fiber systems, and wherein two or more of the at least three fiber systems are secured to each other at one or more of the contact points. A preferred embodiment uses yarn fibers, and art-recognized methods for securing yarn fibers at contact points include but are not limited to ultrasonication, infrared irradiation, and heat exposure.

The at least three fiber systems in at least one of the upper, medial, and lower layers of the fiber scaffold define a plurality of interstices or pores within the fiber scaffold. In a preferred embodiment, the interstices are about 10 μm to about 250 μm; more preferably the interstices are about 25 μm to about 175 μm; and the most preferred interstices are about 50 μm to about 125 μm. The dimensions of the interstices are optimized for controlled release of drugs residing in the interstices, larger interstices effecting more rapid release than smaller interstices bearing a drug of similar size and properties,

In a drug carrier of the present invention, the fiber scaffold can be designed to promote cell growth into the fiber scaffold. In a preferred embodiment, each of the upper, medial, and lower layers of the fiber scaffold have interstices, the medial layer comprises interstices that are smaller than the interstices present in the upper layer, and larger than the interstices in the lower layer, to thereby facilitate cellular growth into the scaffold. Optionally, growth factors or extracellular matrix molecules are engaged with the scaffold to further promote growth into the scaffold.

The three-dimensional fiber scaffold can comprise three orthogonally woven fiber systems, a plurality of braided fiber systems, a plurality of circular woven fiber systems, or combinations thereof. The construction of the fiber systems contributes to the form and/or three-dimensional shape of the drug carrier. In one embodiment, the drug carrier comprises a fabric. In another preferred embodiment, the drug carrier comprises a tube or a “stick”, such as can find application as a stent in an appropriate medical application. Any suitable shape or formation is envisioned in accordance with the present invention, and preferably, the shape promotes efficient drug release.

The drug carriers of the present invention are strong, yet flexible, thus reducing pain and discomfort for the subject while providing a desired profile for drug delivery. The drug carriers of the present invention can be used in a variety of medical applications, including the treatment of small wounds and large wounds (e.g. ≧10 cm×10 cm), in the replacement of bone and related tissues, in other functional tissue replacement applications, in the placement of a stent, in treatment of arthritis, and in contraceptive applications. The ability to customize porosity and free volume along with the unique cross-sections of fibers provided in accordance with the present invention facilitate drug delivery as well as cell proliferation and tissue ingrowth via channeling. These aid in angiogenesis, fibroplasia, and other mechanisms necessary for wound healing and bone grafting. Fast tissue incorporation also reduces or eliminates hematoma, seroma or fistula formation, as well as infection.

Because of (a) the unique porous structures, and (b) exceptional strength properties, the use of a totally absorbable scaffold in drug delivery is now provided by the drug carrier of the present invention. Correspondingly, shrinkage of the present inventive drug carrier is much less as compared to prior art structures. The controlled pore size and pore fraction of the present inventive drug carrier coupled with the controlled geometry of the individual fibers and the controlled rate of bio-degradation also allow for site-specific delivery of chemicals in a variety of medical applications, including in treatment of diseases or infections, treatment of a wound or injury, in the replacement of bone and related tissues, in other functional tissue replacement applications, in the placement of a stent, in treatment of arthritis, and in contraceptive applications. Indeed, the treatment of any medical condition that can be ameliorated by the delivery of a drug via a drug carrier of the present invention is provided in accordance with the present invention.

The thickness of the various layers, and thereby of the entire scaffold, can be altered and customized to fit many medical indications. The drug carriers of the present invention thus constitute a new generation of lightweight, high-strength meshes for delivering drugs. Temporary use of a drug carrier of the present invention is also feasible and entails a totally tightly woven or laminated/coated scaffold to prevent tissue ingrowth. A drug carrier of the present invention can also be fashioned into a dressing, bandage or other desired structure, depending on the envisioned medical application.

When employed, the methods and products of the present invention thus provide a variety of economic benefits, including reducing of medical costs, decreasing number of days of missed work, and prolonging life, among other benefits.

The present co-inventors have developed a new three-dimensional fiber scaffold, wherein the three-dimensional fiber scaffold is formed with fiber systems comprising materials that have been selected to impart improved strength, flexibility and resorbability characteristics to the scaffold. Therefore, a new generation of drug carriers along with methods of making and using the same have been provided in accordance with the present invention.

### B. Drugs Engaged with Scaffold

In a preferred embodiment of the present invention, a drug engaged with the scaffold. Optionally, a drug carrier is engaged with two or more drugs. In this case, each drug can be separately engaged with the scaffold and can occupy different or overlapping positions within the scaffold.

A drug is exemplified by but not limited to an imaging agent, a therapeutic compound, an anti-infective compound, a metabolism-altering compound, a cell growth modulator, a biologically relevant peptide, a gene therapy vector or vector encoding a biologically relevant peptide, and combinations thereof. In one embodiment, the therapeutic compound is selected from the group including, but not limited to, a hormone, insulin, an antidepressant, an antipsychotic, a beta-blocker, a benzodiazepine, a psychedelic, a nootropic, and combinations thereof.

In another embodiment, the anti-infective compound is selected from the group including, but not limited to, an antibiotic, povidone/iodine, silver, silver oxide, other silver salt, a copper salt, sulfadiazine, chlorhexidine, triclosan, cetyl ammonium chloride, cetyl ammonium bromide, a quaternary amine, an alkyl sulfonate, and combinations thereof. In yet another embodiment, the metabolism-altering compound is a steroid hormone or a stimulant. In another embodiment, the cell growth modulator is selected from the group including, but not limited to, a growth factor, a cytokine, a chemokine, a collagen, gelatin, laminin, fibronectin, thrombin, lipids, cartilage oligomeric protein (COMP), thrombospondin, fibrin, fibrinogen, Matrix-GLA (glycine-leucine-alanine) protein, chondrocalcin, tenascin, a mineral, an RGD (Arginine-Glycine-Aspartic Acid) peptide or RGD-peptide containing molecule, elastin, hyaluronic acid, a glycosaminoglycan, a proteoglycan, water, an electrolyte solution, and combinations thereof.

A vector can be used in gene therapy methods. The vector is purified to sufficiently render it essentially free of undesirable contaminants, such as defective interfering adenovirus particles or endotoxins and other pyrogens such that it does not cause any untoward reactions in the individual receiving the vector construct. A preferred technique for purifying the vector involves the use of buoyant density gradients, such as cesium chloride gradient centrifugation. The vector can comprise a virus, including a retroviral vector, adenoviral vector or vaccinia virus whose genome has been manipulated in alternative ways so as to render the virus non-pathogenic. Methods for creating such a viral mutation are detailed in U.S. Pat. No. 4,769,331. Exemplary gene therapy methods are described in U.S. Pat. Nos. 5,279,833; 5,286,634; 5,399,346; 5,646,008; 5,651,964; 5,641,484; and 5,643,567.

In the case of a gene therapy approach, a liposome comprising the gene therapy vector can also be employed. The term “liposome” is meant its art recognized meaning to a particle having one or more encapsulating membranes formed by amphiphilic molecules (such as lipids for example) and in particular particles having a bilayer membrane and an enclosed aqueous core, are versatile carriers for the site specific delivery of therapeutic agents. Other drugs can also be incorporated into a liposome. Liposomes themselves are spherical vesicles having a lipid layer surrounding a central space. The present invention is particularly concerned with unilammellar and multilamellar liposomes which respectively have a single lipid bilayer or multiple lipid bilayers surrounding an aqueous core. Liposomes spontaneously form upon dispersion of lipids, particularly phospholipids, in aqueous media and the liposomal structure of the agents of the invention can be produced by conventional techniques. Such conventional techniques are referred to in WO92/21017 (Unger) and by Papahadjopolous in Ann Rep. Med. Chem. 14: 250-260 (1979) and include reverse evaporation, freeze-thaw, detergent dialysis, homogenization, sonication, microemulsification and spontaneous formation upon hydration of a dry lipid film. Multi-lamellar liposomes can be used according to the invention or can be converted to liposomes with lower lamellarity, or to unilamellar liposomes, by known methods. Unilamellar liposomes can also be prepared directly.

Exemplary imaging agents include, but are not limited to, paramagnetic, radioactive and fluorogenic ions. Preferably, the imaging agent comprises a radioactive imaging agent. Exemplary radioactive imaging agents include, but are not limited to, gamma-emitters, positron-emitters and x-ray-emitters. Particularly contemplated radioactive imaging agents include, but are not limited to, ⁴³K, ⁵²Fe, ⁵⁷Co, ⁶⁷Cu, ⁶⁷Ga, ⁶⁸Ga, ⁷⁷Br, ⁸¹Rb/^{81M}Kr, ^{87m}Sr, ^{99m}Tc, ¹¹¹In, ¹¹³In, ¹²³I, ¹²⁵I, ¹²⁷Cs, ¹²⁹Cs, ¹³¹I, ¹³²I, ¹⁹⁷Hg, ²⁰³Pb and ²⁰⁶Bi. Other radioactive imaging agents known by one skilled in the art can be used as well.

The drug can be substantially pure, solubilized in an appropriate solvent, present as a slurry or mixture, encapsulated within a coating, or formulated in any other effective manner. Formulation of the drug is selected to be appropriate with methods for engaging the drug with the fiber scaffold and proposed disengagement of the drug from the same. In one embodiment, the drug is solubilized in a biocompatible solvent. In another embodiment, the drug is encapsulated in microspheres or liposomes.

Techniques for engagement of the drug with the scaffold include but are not limited to impregnating, coating, filling, adhering, attaching, incorporating during synthesis, and loading drug fibers. Preferred techniques are described immediately below.

The drug can be impregnated within the entire scaffold, within a single layer of the scaffold, or within an individual fiber system of the scaffold. To impregnate an entire scaffold, a drug can be injected into the scaffold using an injection technique disclosed in U.S. Pat. No. 5,770,417, the entire contents of which are herein incorporated by reference. In another embodiment, the scaffold can be submerged in a solution containing the drug such that the drug fills the interstices within the scaffold.

To impregnate a single fiber or layer of fibers, a fiber is swollen to open fiber pores, a drug is loaded into the opened pores, and the fiber is then collapsed to its original dimensions. For example, a fiber comprising a coating of poly-L-lactic acid and poly-caprolactone can be swollen in 40% trifluoroacetic acid with polyethylene oxide, liposomes containing a drug compound can occupy the swollen fiber, and the liposomes are captured within the fiber when the fiber is returned to its pre-swollen dimensions. Representative “swelling techniques” are disclosed in U.S. Pat. No. 5,980,551, the contents of which are herein incorporated by reference.

In another embodiment, the drug is coated on the entire scaffold, on a single layer of the scaffold, or on an individual fiber system of the scaffold. A method for coating a solid form with a drug is disclosed in U.S. Pat. Nos. 5,980,551 and 5,876,452, the entire contents of each patent being herein incorporated by reference. Briefly, a scaffold, or portion thereof, is immersed in a solution containing the drug, and the solvent is allowed to evaporate, thereby precipitating the drug on the surface of the scaffold.

In yet another embodiment, the drug is loaded into a hollow fiber via an open end of the hollow fiber. Loading of the hollow fiber can be accomplished by pouring or injecting a drug in liquid form into the interior of the fiber. Alternatively, a vacuum mechanism can be used to load the drug into a hollow fiber, as disclosed in U.S. Pat. No. 5,538,735, the entire contents of which are herein incorporated by reference. Briefly, a hollow fiber is submerged in a solution containing a drug. The submerged fiber is placed in a vacuum chamber, and air pressure within the chamber is reduced, thereby drawing air out of the hollow fiber. The air pressure is then restored, and the liquid containing the drug is drawn into the hollow fiber.

In still another embodiment, the drug is adhered to the entire scaffold, wherein the drug is adhered to a single layer of the scaffold, or the drug is adhered to the an individual fiber system of the scaffold. For example, Kwok et al., (1999), J of Controlled Release 62:301-311, herein fully incorporated by reference, demonstrates preparation of a substrate for engagement with a compound by creating a radio frequency-plasma-deposited n-butyl methacrylate overlayer having highly reactive species at the surface. These “sticky reactive groups” promote bonding of a desired compound to the substrate. In yet a further embodiment, the drug is incorporated within a fiber system during a fiber-synthesizing step. A method for incorporating a bioactive agent into a polymeric material is disclosed in U.S. Pat. No. 5,876,452, the contents of which are herein fully incorporated by reference. Briefly, the polymer is solubilized, and a bioactive agent is mixed with the solubilized polymer. The solvent is removed, preferably by precipitation, and the polymer is pressed into a mold to confer a shape, such as any desired shape of a fiber. The above steps are performed at a temperature below the degradation temperature of the bioactive agent, so as to maintain biological activity of the agent.

In still a further embodiment, the fibers of at least one of the fiber systems comprises a drug. For example, the fibers of at least one of the fiber systems can comprise hyaluronic acid or collagen. Thus, the scaffold itself can be woven, braided, or otherwise prepared using a fiber comprising a drug.

The drug can be engaged with the scaffold such that it is unevenly distributed within the scaffold. In one embodiment, a gradient of drug concentration is created by engaging fibers residing in successively adjacent positions along any one dimension of the scaffold with progressively decreasing amounts of the drug. In another preferred embodiment, the drug is engaged with alternating fiber layers within the scaffold. For example, the upper and lower layers of a three-layer scaffold can be engaged with the drug, while the medial layer of the same three-layer scaffold is not engaged with the drug. In another embodiment, the drug carrier is designed such that the drug is engaged with fibers that reside at internal positions of the three-dimensional scaffold, and the drug is not engaged with fibers that reside at superficial positions within the scaffold. Alternatively, the drug can be constructed wherein the drug is engaged with fibers that reside at superficial positions within the three-dimensional scaffold, and the drug is absent from fibers that reside at internal positions of the scaffold.

The drugs can be formulated according to art-recognized techniques. A good discussion of formulation techniques can be seen in U.S. Pat. No. 4,229,447 issued Oct. 21, 1980 to Porter and U.S. Pat. No. 5,504,086 issued Apr. 2, 1996 to Ellinwood and Gupta. A good discussion of formulation for transdermal administration can be seen in U.S. Pat. No. 5,016,652 issued May 21, 1991 to Rose and Jarvik.

Formulations can comprise appropriate fluids such as but not limited to, water, rehydration solutions (i.e., water with electrolytes such as potassium citrate and sodium chloride, for instance the solution available under the trade name RESOL® from Wyeth Laboratories), nutritional fluids (i.e., milk, fruit juice), and combinations thereof. Hence, it is also contemplated that additional ingredients, such as various excipients, carriers, surfactants, nutriments, and the like, as well as various medicaments, other than the drug can be present in the formulation. Medicaments other than the candidate drug for delivery can include, but are not limited to, osmolytic polyols and osmolytic amino acids (i.e., myo-inositol, sorbitol, glycine, alanine, glutamine, glutamate, aspartate, proline, and taurine), cardiotonics (i.e., glycocyamine), analgesics, antibiotics, electrolytes (i.e., organic or mineral electrolytes such as salts), and combinations thereof.

### C. Drug Release

Disengagement of the drug from the scaffold can occur by diffusion or leaching of the drug from the interstices the scaffold, or portion thereof; diffusion or leaching from a hollow fiber via an open end of a hollow fiber; solubilization of a drug-coated surface of the scaffold, or portion thereof; detachment of the drug from a site of adherence to the scaffold, or portion thereof; or dissolution of a drug-constituted fiber.

In one embodiment, drug release from the drug carrier occurs via diffusion or leaching from the interstices of the scaffold. Interstices can have macroscopic or molecular dimensions, and preferably, the pore size is optimized for drug release. In a further embodiment, pores are enlarged sufficient to permit drug traversal by swelling of the scaffold, or portion thereof, during use. In another embodiment, the pores comprise an open end or ends of a hollow fiber. In yet another embodiment, pores are created by the decomposition of the fiber. In still another embodiment, the decomposition of a fiber system occurs at a rate inversely proportional to a rate of ingrowth of cells near a carrier that has been implanted or injected into a subject.

In another embodiment, disengagement of the drug from a drug-coated scaffold occurs by solubilization of the coating. In another embodiment, disengagement of the drug from the scaffold, occurs via detachment from sites of adherence to a fiber system. In another embodiment, disengagement of the drug from the scaffold occurs by dissolution of a fiber constituted of the drug. For example, a collagen fiber can dissolve into constituent collagen units. Drug release can also be accomplished through local conditions at the site of use, including but not limited to solvent, pH, cellular setting and in vivo enzymatic cleavage. Alternatively, a trigger or cue can be employed to initiate release. For example, heat or electrical current can be applied externally at the site of implantation of the drug carrier to initiate drug release, such by heat-induced cleavage of a chemical linkage. Application of heat is envisioned to be particularly appropriate in the context of delivery of an anti-arthritic agent.

In the context of each mechanism for drug release from the carrier, the rate of drug release is influenced by provision of a solvent. For use of the drug carrier in vivo, a solvent is provided by placement of the drug carrier in a subject. In another embodiment, a solvent can be applied to a drug carrier to trigger drug release, thereby “activating” the drug carrier. Subsequently, the activated drug carrier can be applied to an external body site for drug delivery.

### D. Drug Release Profiles

Another aspect of the drug carrier of the present invention pertains to disengagement of the drug from the scaffold according to a controlled release profile. The spatial, temporal, and dose properties of drug release are predetermined by selecting an appropriate fiber composition, an appropriate construction of the three-dimensional scaffold, and an appropriate method for engaging a drug with the scaffold, in the context of its intended use. An aspect of the invention is versatile design to effect acute release, continuous release, intermittent release, and combinations thereof.

In one embodiment, the drug carrier mediates immediate and acute release. For example, a scaffold can be coated with a drug, wherein placement of the carrier in a solvent or in vivo results in rapid solubilization of the drug. Immediate and acute release can also be accomplished by constructing a drug carrier comprising a three-dimensional fiber scaffold, wherein the fibers of the fiber scaffold comprise a material that demonstrates rapid swelling upon exposure to a solvent or in vivo conditions. A drug is incorporated within the fiber, and placement of the carrier leads to rapid transfer of the drug from the carrier to the subject. Immediate and acute release can also be accomplished by constructing a drug carrier comprising a three-dimensional fiber scaffold, so that the fiber scaffold is built of hollow fibers having a substantially large interior diameter. In this case, a drug is loaded within the hollow interior of the fibers, and placement of the scaffold effects immediate and rapid diffusion of the drug through an open end of the hollow fiber. Therefore, it is an aspect of the invention that a same desired drug release profile can be accomplished by a drug carrier of variable construction.

In another embodiment, a drug carrier of the present invention mediates acute release following a delay. For example, the scaffold can be constructed of absorbable fibers, wherein fibers residing at internal positions of the scaffold are engaged with a drug. Fibers residing at superficial positions of the scaffold lack the drug. Following implantation of a drug carrier so constructed, a temporal period elapses wherein the superficial drug-deficient fibers are absorbed. A period of drug release that corresponds to absorption of the drug-loaded fibers follows.

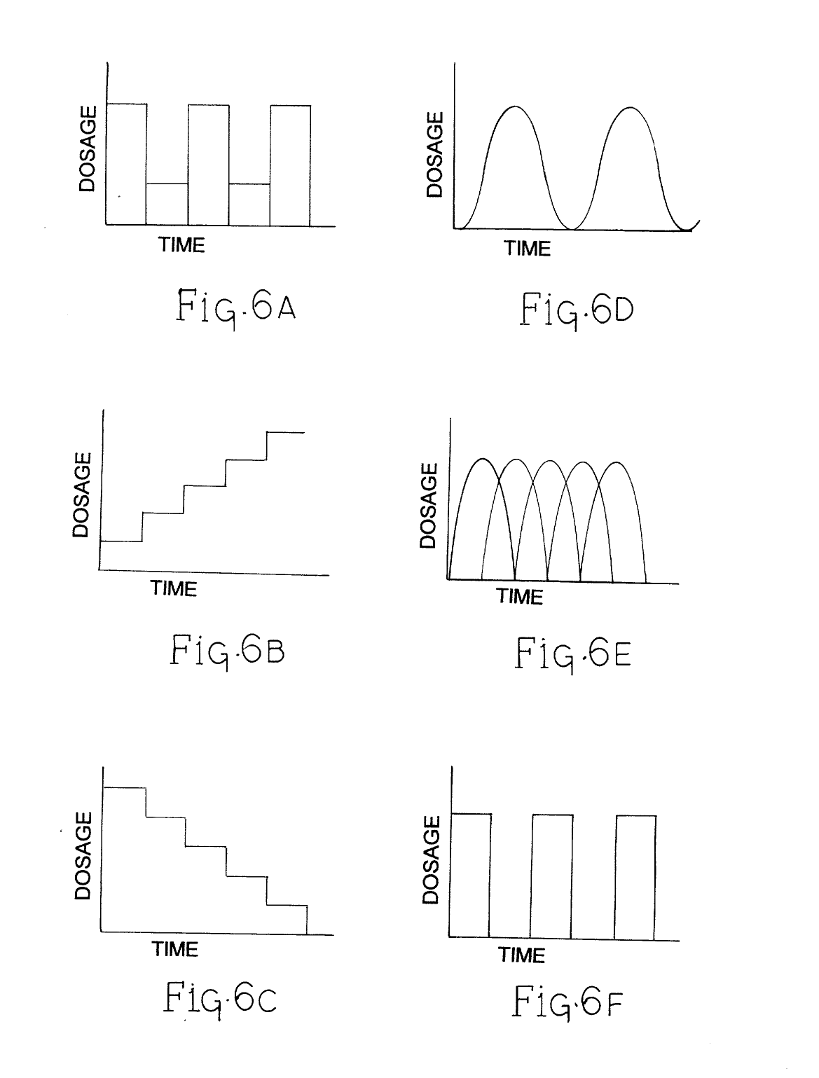

In yet another embodiment, a drug carrier of the present invention is constructed of absorbable fibers, and a drug is incorporated into all fibers of the scaffold. A drug carrier so constructed can be used for continuous delivery, with the drug released at a steady rate concomitant with absorption of the fiber scaffold. A representation of this profile is set forth in FIG. 6E.

In still another embodiment, a drug is engaged with the scaffold of a drug carrier of the present invention such that a concentration of the drug is variable along one or more dimensions. For example, a gradient of drug concentration can be created by engaging fibers that are in successively adjacent positions along any one dimension of the scaffold with progressively decreasing amounts of a selected drug. In a preferred embodiment, a drug carrier comprising a three-dimensional fiber scaffold is constructed wherein each layer of the scaffold has interstices. The plurality of layers also further comprises an upper layer, a medial layer and a lower layer. The interstices of the medial layer are smaller than the interstices of the upper layer, and the interstices of the lower layer are smaller than the interstices of the medial layer. In this case it is also preferred that all interstices bear the selected drug. A drug carrier so constructed demonstrates a rate of drug release from the upper layer that is greater than a rate of drug release from the medial layer, and a rate of drug release from the medial layer that is greater than a rate of drug release from the lower layer. Contact of a subject at the upper layer of a drug carrier so constructed elicits a drug release profile characterized by continuous drug release with progressively decreasing dose. Alternatively, contact of a subject at the lower layer of a drug carrier so constructed effects a drug release profile characterized by continuous drug release with progressively increasing dose. Representations of these step-wise profiles are set forth in FIGS. 6B and 6C.

In another embodiment, disengagement of the drug from the scaffold is characterized by intermittent drug release. As discussed above, the term “intermittent release”, as used herein, is two or more temporal periods of a first amount of drug release, wherein the first amount can be the same or different, interluded by a temporal period of a second, different amount of drug release. For example, the term “intermittent release” can refer to two or more temporal periods of drug release interluded by a temporal period substantially without drug release (e.g. FIG. 6F). Alternatively, the term “intermittent release” can refer to a first temporal period of a first amount of drug release, followed by a second temporal period with a second, different amount of drug release, followed by a third temporal period with still another third, different amount of drug release. The term “intermittent release” can also be used synonymously with “cyclic release” or “non-constant release”. Additional representations of “intermittent” release are set forth in FIGS. 6A and 6D.

By way of additional example, a drug carrier of the present invention can be constructed of absorbable fibers wherein an upper layer and a lower layer are engaged with a drug, and a medial layer there between is not engaged with the drug. A drug carrier so constructed defines three temporal periods when contacted with a subject at its upper layer. A first temporal period is characterized by drug release concomitant with absorption of the upper layer, followed by a second temporal period characterized by lack of drug release concomitant with absorption of the medial layer, in turn followed by a third period characterized with drug release concomitant with absorption of the lower layer. Therefore, it is an aspect of the invention to provide a drug carrier that can be used to administer successive drug doses that are temporally non-overlapping.

In yet another embodiment, a drug carrier of the present invention is constructed to enable directional drug release. For example, a drug carrier can comprise a scaffold of three layers, an upper layer, a medial layer, and a lower layer. In this case, the lower layer further comprises a outer surface that is substantially impermeable to drug movement. Optimally, a drug is coated on each fiber of the scaffold. Solubilization of the coated drug is initiated, and the drug diffuses from the scaffold at an interface bounded by the upper layer of the scaffold. The drug is unable to traverse the outer surface of the lower layer. Once the upper layer is cleared of the drug, the drug content of the medial layer, and subsequently the drug content of the lower layer, diffuses to the upper layer, and also transfers from the carrier through the upper layer.

In still another embodiment, a drug carrier of the present invention comprises two or more drugs. The two or more drugs are engaged within the same scaffold, wherein each drug is disengaged from the scaffold according to a different controlled release profile. For example, a drug carrier can be constructed, wherein all fiber systems of a three-dimensional fiber scaffold comprise an absorbable material, and wherein drug A is coated on the surface of each fiber, and drug B is incorporated within each fiber during synthesis of the fiber. Implantation of a drug carrier so-constructed initiates rapid release of drug A by solubilization of the drug coating, and onset of slower, continuous release of drug B as the scaffold fibers are absorbed.

In summary, any combination of spatial, temporal, and/or dose properties of drug release is provided by the present inventive drug carrier through the selection of an appropriate fiber composition, an appropriate construction of the three-dimensional scaffold, and an appropriate method for engaging a drug with the scaffold, in the context of its intended use. Amounts of drug that is delivered can vary upwardly or downwardly as desired over a series of temporal periods. Amounts of delivered drug can be maintained at constant levels over a temporal period. Two or more different drugs can be delivered at varying amounts over a series of temporal periods. The versatility of the present inventive drug carrier is thus believed to be demonstrated.

### E. Method of Producing a Drug Carrier

A method for producing a drug carrier article is also disclosed. The method comprises forming a scaffold comprising a plurality of layers and a fiber system interconnecting the plurality of layers, wherein the layers define internal and superficial positions of the scaffold. Further, as described in detail herein, a drug is engaged with the scaffold, such as by loading a hollow fiber with the drug, by providing a fiber that is made of a drug composition, and/or by otherwise engaging the drug with the scaffold. Engagement of the drug with the scaffold is provided so that the drug disengages from the scaffold according to a controlled release profile, thereby transferring the drug from the carrier to a subject.

In the method, the scaffold can be formed by lamination of layers, which can be accomplished via heating, adhesives, combinations thereof, or in another suitable manner. However, in a preferred embodiment, the method comprises forming a three-dimensional fiber scaffold with at least three fiber systems such that two of the three fiber systems the plurality of layers, wherein one of the at least three fiber systems interconnects the plurality of layers, and wherein the three dimensions of the scaffold define internal and superficial positions within the scaffold. Further, a drug is engaged with the scaffold. Engagement of the drug with the scaffold is provided so that the drug disengages from the scaffold according to a controlled release profile, thereby transferring the drug from the carrier to a subject.

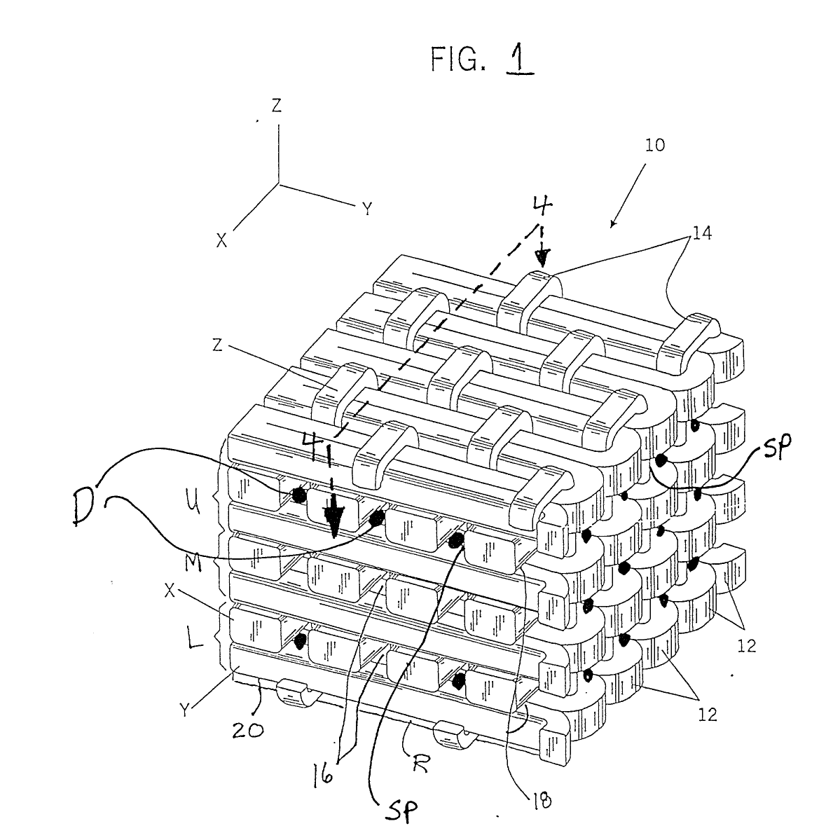

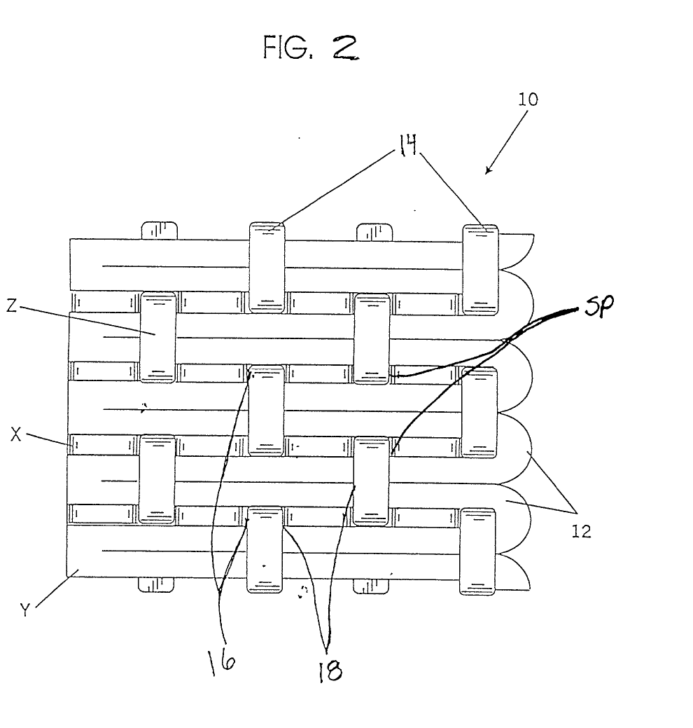

Referring now to FIGS. 1-4, applicants wish to describe an embodiment of a three-dimensional fiber drug carrier of the present invention, as well as methods of making the same. While FIGS. 1-4 illustrate orthogonally woven three-dimensional fiber scaffolds, the invention is not intended to be limited to this structure but to include other three-dimensional fiber scaffolds formed of at least three systems of fibers so as to preferably provide interstices within the structure. These structures can include woven, braided, circular woven and knitted three-dimensional structures formed of at least three different fiber systems.

Well recognized methods for making three-dimensional structures comprising at least three fiber systems are generally applicable to the process of making a scaffold of the present invention. For example, the 3-D fabric can be fabricated on one of the 3-D weaving machines located at 3TEX, Inc., Cary, N.C. Representative methods of making three-dimensional textile structures are also disclosed in U.S. Pat. No. 5,465,760 issued to Mohamed et al. on Nov. 14, 1995 and U.S. Pat. No. 5,085,252 issued to Mohamed et al. on Feb. 4, 1992, and the contents of each of these U.S. patents are herein incorporated by reference.

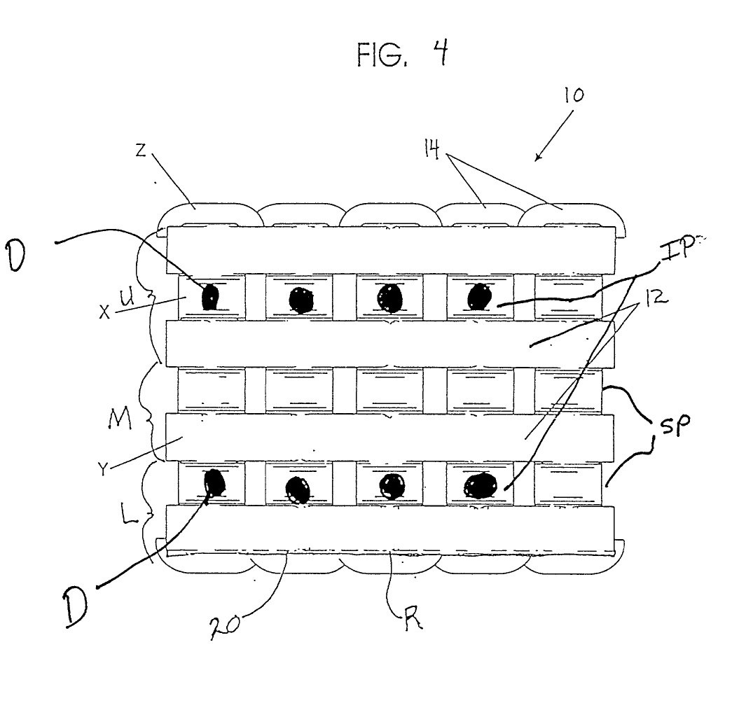

Referring now to FIGS. 1-4, a volumetric section of a first embodiment of a three-dimensional woven scaffold according to the present invention is shown and generally designated 10. Three-dimensional textile scaffold 10 preferably comprises at least three primary systems of fibers. A first system includes a plurality of x-fibers (or warp fibers) X running straight and in a spaced parallel relation along the x-axis. A second system includes a plurality of y-fibers (or fill or weft fibers) Y running straight and in a spaced parallel relation along the y-axis. In scaffold 10, y-fibers Y are actually one or more single, continuous fibers that are extended in one direction along the y-axis across the plane defined by the first system of x-fibers, and then made to reverse direction in a repeated manner around loops or curved sections 12 at the fold of scaffold 10 so as to extend across the plane of the first system in the opposite direction. It is preferable that x-fibers X and y-fibers Y, and thus the first and second systems, be disposed in a mutually orthogonal relation, such that the x- and y-axes are defined as in a Cartesian coordinate system. Internal positions IP (best seen in FIG. 4) and superficial positions SP are defined by and within scaffold 10.

Continuing with FIGS. 1-4, a third system includes a plurality of z-fibers Z running in parallel relation through the planes of x-fibers X and y-fibers Y, such that z-fibers Z can be said to interconnect or bind the first and second systems and, in the case of a multiple-layered scaffold, to interconnect or bind all layers forming scaffold 10. Preferably, z-fibers Z generally extend along the Cartesian z-axis such that z-fibers Z are mutually orthogonal to both x-fibers X and y-fibers Y or, stated differently, the third system is preferably disposed in an out-plane that is perpendicular to the in-plane defined by the first and second systems. Alternatively, or in addition to the orthogonal z-fibers Z, scaffold 10 can include fibers running along a bias direction, or a direction angled with respect to the Cartesian axes. It is further preferable that z-fibers Z comprise one or more fibers which extend through the first and second systems in one direction along the z-axis and reverse direction in a repeated manner around curved sections 14 at the edge of scaffold 10.

Continuing with FIGS. 1-4, fiber systems X and Y define an upper layer U, a lower layer L, and a medial layer M between upper layer U and lower layer L, within the three-dimensional scaffold 10. The terms “upper layer”, “medial layer”, and “lower layer” have been adopted to facilitate description of scaffold 10, and upon application to a subject, lower layer L comprises an outer side of scaffold 10. Each layer can be defined as including one system of x-fibers X and one system of y-fibers Y, except for the outermost surface layers where only y-fibers Y are present. The actual number of layers, and the number of individual fiber systems included within each layer, will depend upon the desired thickness of the finished scaffold. Fiber system Z interconnects upper layer U, lower layer L and medial layer M.

Continuing with FIGS. 1-4, fiber systems X, Y and Z are interlaced so as to provide a plurality of pores or interstices 16 within textile scaffold 10. Optionally, scaffold 10 is not crimped so that interstices 16 remain intact after the intermeshing of fiber systems X, Y and Z. Preferably, fiber systems X, Y and Z are secured to each other at one or more contact points 18 to facilitate maintenance of interstices 16 while also providing cuttability and suturability. The securing or setting of fiber systems X, Y and/or Z at a contact point 18 can be accomplished by any suitable technique, such as sonication or heat molding. The sizes of the pores or interstices can range from about 10 μm to about 250 μm, can more preferably range from about 25 μm to about 175 μm, and the most preferred pore size of ranges from about 50 μm to about 125 μm. In scaffold 10, interstices 16 are of a substantially uniform size throughout each of upper layer U, medial layer M and lower layer L. However, scaffold 10 can also be designed such that medial layer M comprises interstices 16 that are smaller than the interstices 16 present in the upper layer, and larger than the interstices 16 in the lower layer, to thereby facilitate cellular growth into scaffold 10 when such growth is desirable. In this case, growth factors or extracellular matrix molecules can engaged with scaffold 10 to further promote growth into scaffold 10.

Continuing with FIGS. 1-4, a drug D is releasably engaged with scaffold 10 according to a controlled release profile. For example, the dimensions of interstices 16 can be optimized for controlled release of a drug D residing in interstices 16, i.e. larger interstices effect more rapid release than smaller interstices bearing a drug of similar size and properties. Drug D can also be engaged with scaffold 10 such that a concentration of drug D is variable along one or more dimensions of a fiber system X, Y, or Z. As best seen in FIG. 4, drug D is engaged with fibers residing at internal positions IP of scaffold 10, and at superficial positions SP of scaffold 10. Optionally, depending on the desired release profile as disclosed herein, drug D can be engaged at internal positions IP of scaffold 10 but not at superficial positions SP, or at superficial positions SP but not at internal positions IP.

Continuing with FIGS. 1-4, drug D is engaged with alternating fiber layers within the scaffold 10. As shown in FIGS. 1-4, for example, upper and lower layers U and L of scaffold are engaged with a drug, and medial layer M is not engaged with drug D. Optionally, all layers U, L and M can be engaged with a drug, or only one of the layers U, L and M can be engaged with a drug. Thus, drug D can be engaged in upper layer U only, or in medial layer M only, or in lower layer L only.

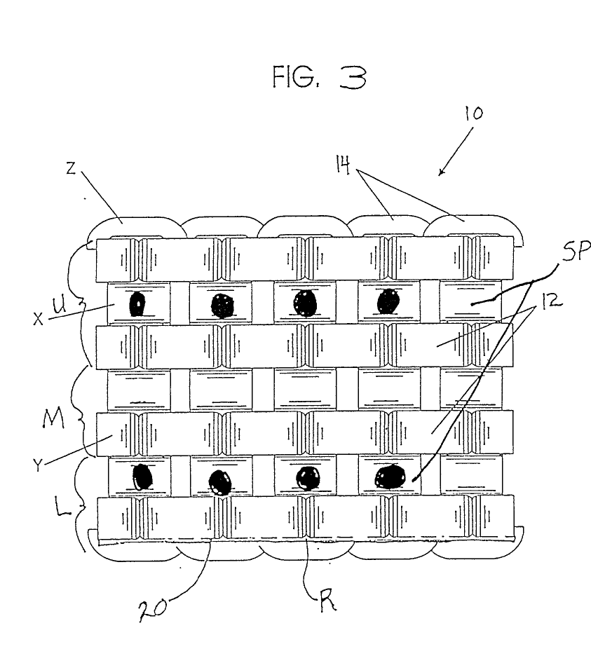

As best seen in FIGS. 3 and 4, an outer surface 20 of lower layer L of textile scaffold 10 has no pores or interstices. This can be accomplished by coating outer surface 20 with a resin material R, preferably to the extent that resin material R coats all of outer surface 20. Alternatively, outer surface 20 can be covered with an inert material, e.g. polytetrafluoroethylene (PTFE) such as that sold under the registered trademark TEFLON® by E. I. DuPont de Nemours & Co. For example, outer surface 20 can be covered with a sheet of PTFE. As an additional alternative, outer surface 20 can be tightly woven with substantially no pores. Thus, outer surface 20 can optionally comprise a sealed surface or a smooth sealed surface. In this embodiment, outer surface 20 of lower layer L is substantially impermeable and thereby blocks drug delivery through outer surface 20 to provide directional drug release.

Properties of scaffold 10 can further be enhanced with controlled cellular matrix formation within resin material R. Briefly, a foamable polymer resin material R is applied to a portion of three-dimensional textile scaffold 10 so as to fill interstices 16 and impregnate a portion of the three-dimensional textile scaffold 10. Then, foamable polymer material R is foamed to produce a cellular foamed polymer matrix material R containing a plurality of voids or cells distributed substantially throughout the material R. Preferably, the cellular matrix within material R comprises bubbles defining a void diameter of between about 0.01 μm to 10.0 μm, but cell void diameter can be larger. The dimensions of the voids can be optimized for controlled release of a drug residing in the voids. Thus, drug D can also be loaded in the voids of a foamed polymer resin R. The foamed polymer resin R also increases the strength and stiffness properties of the material per unit weight by intentionally creating defined voids throughout the composite structure. The enhanced biomechanical strength provided by this approach makes this embodiment of a drug carrier of the present invention particularly suitable for application in bone repair (especially fractured bones) or other orthopedic medical applications.

As an alternative to the application of resin material R, a portion of scaffold 10 of the present invention can be heat-molded if the portion of scaffold 10 comprises thermoplastic fibers. As part of this process the thermoplastic portion can be foamed directly to provide a cellular matrix therein.

Representative foaming techniques are also disclosed in U.S. Pat. No. 3,796,779 issued to Greenberg; U.S. Pat. No. 4,473,665 to J. E. Martini-Vredrensky et al.; U.S. Pat. No. 4,761,256 to Hardenbrook et al.; and U.S. Pat. No. 5,334,356 and U.S. Pat. No. 5,158,986 both issued to Baldwin et al. The entire contents of each of these U.S. patents are herein incorporated by reference.

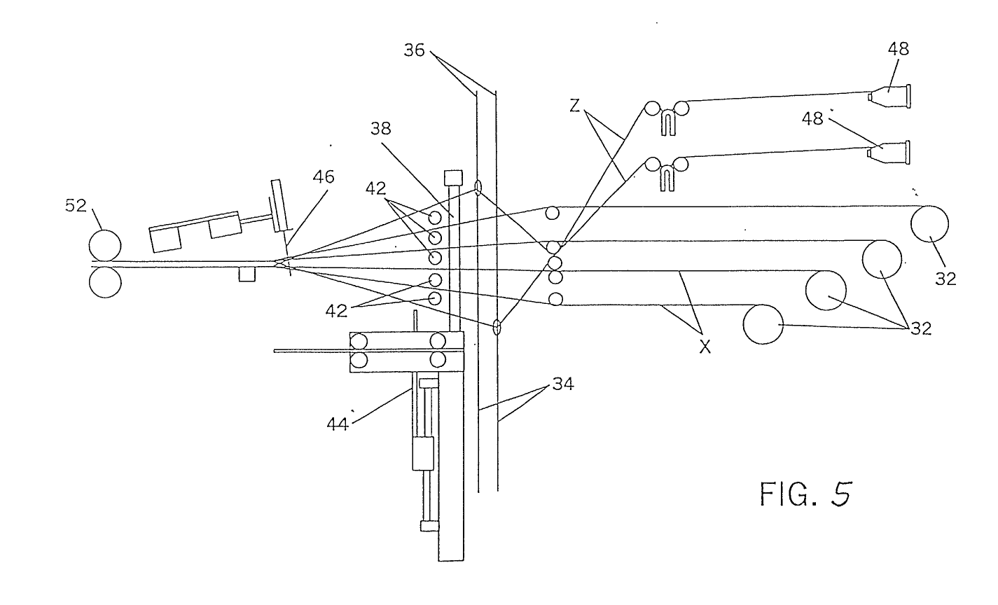

The process by which scaffold 10 is formed will now be further described with reference to the schematic shown in FIG. 5. Lengthwise or x-fibers X are drawn under tension from an x-fiber feeding device 32 such as a set of warp beams (as shown) or a creel (not shown), between heddles 34 of harnesses 36, and through a beat-up reed 38, thereby forming systems of x-fibers X which are in horizontal and vertical alignment. Crosswise or y-fibers Y (not shown) are inserted between the systems of x-fibers X using fill insertion rapiers 42. Preferably, all y-fibers Y are inserted simultaneously in order to guarantee their straightness within the core of finished scaffold 10 and to increase productivity. Beat-up reed 38 is actuated to apply force on y-fibers Y as scaffold 10 is being formed, thereby packing x-fibers X and y-fibers Y into a structure having interstices or pores of a desired pore size. It will be understood that other devices, such as a conventional selvedge hold device 44 (for preventing the selvedge or edge on either side of the fabric from unraveling) and fill hold device 46 (for tensioning of y-fibers Y), are preferably employed in known manner during the forming of scaffold 10.

Z-fibers Z are drawn under tension from a z-fiber feeding device 48 such a creel with bobbins (as shown) or one or more beams (not shown), and inserted through the layers formed by the systems of x-fibers X and y-fibers Y under the control of harnesses 36 with cross-moving heddles 34 and beat-up reed 38. Take-up roll 52 is used to advance scaffold 10 forwardly. One specific and exemplary process and apparatus that can be utilized to form a structure such as scaffold 10 is described in detail in U.S. Pat. No. 5,085,252 to Mohamed et al., which applicants incorporate herein by reference.

The thickness and composition of the layers of scaffold 10, and thereby of the entire structure, can be altered and customized to fit a variety of drug delivery applications. A representative thickness is of a 2-warp/3-fill configuration. Additional fiber systems X and Y can be included within any of upper layer U, lower layer L and medial layer M of textile scaffold 10. For example, (+)/(−) bias fibers can be incorporated within textile scaffold 10 in accordance with techniques described in U.S. Pat. No. 5,465,760. Thus, textile scaffold 10 having more than three fiber systems are also provided in accordance with the present invention, including textile scaffolds having four and five fiber systems. The additional fiber systems can comprise absorbable materials, non-absorbable materials, or combinations thereof, depending on the particular application for the scaffold.

In one embodiment of the scaffold 10 of the present invention, the fiber systems X and Y which define upper layer U comprise primarily an absorbable material as defined herein; the fiber systems X and Y which define lower layer L comprise primarily a non-absorbable material as defined herein; and the fiber systems X and Y which define medial layer M comprise both absorbable and non-absorbable materials as defined herein. Fiber systems X and Y which define upper layer U can also comprise a relatively smaller proportion of non-absorbable material. Correspondingly, fiber systems X and Y which define lower layer L can also comprise a relatively smaller proportion of an absorbable material. This construction can be accomplished by incorporating more filaments of an absorbable material as compared to non-absorbable material into the fiber systems X and Y which comprise upper layer U; and incorporating relatively more non-absorbable filaments as compared to absorbable filaments into fiber systems X and Y which define lower layer L, as a part of a weaving, knitting, braiding or other process for interlacing fiber systems X and Y to form textile scaffold 10. The filaments can be either twisted, zero-twisted, or both. Multifilament fibers are preferred for flexibility.

Thus, fiber systems X and Y can comprise both an absorbable material and a non-absorbable material in accordance with the present invention, as can fiber system Z. In the fiber systems X and Y that comprise upper layer U, and in fiber system Z, representative proportions of absorbable material to non-absorbable material can comprise 100/0, 90/10, 80/20, 70/30, 60/40, 50/50, 40/60, 30/70, 20/80, 10/90 or 0/100. In a first embodiment, representative proportions of absorbable material to non-absorbable material preferably comprise 100/0, 90/10, 80/20, 70/30 and 60/40. In the fiber systems X and Y that comprise lower layer L, and in fiber system Z, representative proportions of non-absorbable material to absorbable material can comprise 100/0, 90/10, 80/20, 70/30, 60/40, 50/50, 40/60, 30/70, 20/80, 10/90 or 0/100. In a second embodiment, representative proportions of non-absorbable material to absorbable material preferably comprise 100/0, 90/10, 80/20, 70/30 and 60/40. Medial layer M can comprise the noted proportions of either of upper layer U or lower layer L, or can comprise 50/50 absorbable/non-absorbable material. While representative proportions are provided herein, any suitable proportions can be employed in accordance with the present invention, and preferably, proportions are chosen depending on a particular drug delivery application envisioned for the scaffold of the present invention.

### F. Method of Drug/Delivery

A method for delivering a drug to a subject using a drug carrier is also disclosed. The method comprises providing a drug carrier as disclosed herein above; and placing the carrier at a site of desired exposure to the drug. The site of desired exposure to the drug can be an internal body site or an external body site. In a preferred embodiment, the drug carrier is implanted or injected at an internal body site. In another preferred embodiment, the drug carrier is adhered to or wrapped around an external body site.

Contemplated is the delivery of drugs to warm-blooded vertebrates, including mammals such as humans, as well as those mammals of importance due to being endangered (such as Siberian tigers), of economical importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also contemplated is the treatment of birds, including the treatment of those kinds of birds that are endangered, kept in zoos, as well as fowl, and more particularly domesticated fowl, i.e., poultry, such as turkeys, chickens, ducks, geese, guinea fowl, and the like, as they are also of economical importance to humans. Thus, contemplated is the treatment of livestock, including, but not limited to, domesticated swine (pigs and hogs), ruminants, horses, poultry, and the like.

### G. Preferred Applications

In a preferred application of the present invention, a drug carrier is constructed for implantation at a wound or other similar injury. To this end, the fiber scaffold is constructed of fibers comprising an absorbable material, and an antibiotic is incorporated into the fiber scaffold. The drug carrier so constructed is implanted at the site of injury, thereby releasing the antibiotic to prevent bacterial infection at the injury site.

In another application of the present invention, the fiber systems of the drug carrier comprise a non-absorbable fiber, and the fiber scaffold has a cloth or fabric form. A drug is engaged in the interstices of the scaffold, and a drug carrier so-constructed is applied to an external body surface, whereby the drug leaches from the fiber scaffold to the adjacent skin and is delivered via a transdermal route to an underlying site of therapeutic effect. Preferably, the lower layer of the drug carrier further comprises an outer surface that is substantially impermeable to the drug, and the carrier is placed at an external body site such that the upper layer is adjacent to the external body site. The outer surface of the lower layer is spaced away from the external body site surface, so that the drug leaches out only from the upper layer.

The drug carriers of the present invention can also deliver contraceptive agents according to a predetermined, desired profile. Alternatively, the carrier of the present invention can be employed to deliver an anti-arthritic agent. Further, the carrier of the present invention can be fabricated for use as a stent.

The drug carriers of the present invention are strong, yet flexible, thus reducing pain and discomfort for the subject while providing a desired profile for drug delivery. The drug carriers of the present invention can be used in a variety of medical applications, including the treatment of small wounds and large wounds (e.g. ≧10 cm×10 cm), in the replacement of bone and related tissues, and in other functional tissue replacement applications. The ability to customize porosity and free volume along with the unique cross-sections of fibers provided in accordance with the present invention facilitate drug delivery as well as cell proliferation and tissue ingrowth via channeling. These aid in angiogenesis, fibroplasia, and other mechanisms necessary for wound healing, bone grafting and tissue replacement. Fast tissue incorporation also reduces or eliminates hematoma, seroma or fistula formation, as well as infection.

Because of (a) the unique porous structures, and (b) exceptional strength properties, the use of a totally absorbable scaffold in drug delivery is now provided by the drug carrier of the present invention. Correspondingly, shrinkage of the present inventive drug carrier is much less as compared to prior art structures. The controlled pore size and pore fraction of the present inventive drug carrier coupled with the controlled geometry of the individual fibers and the controlled rate of biodegradation also allow for site-specific delivery of chemicals in a variety of medical applications, including in wound or injury repair, in the replacement of bone and related tissues, and in other functional tissue replacement applications.

The thickness of the various layers, and thereby of the entire scaffold, can be altered and customized to fit many medical indications. The drug carriers of the present invention thus constitute a new generation of lightweight, high-strength meshes for delivering drugs. Temporary use of a drug carrier of the present invention is also feasible and entails a totally tightly woven or laminated/coated scaffold to prevent tissue ingrowth. A drug carrier of the present invention can also be fashioned into a dressing, bandage or other desired structure, depending on the envisioned medical application.

When employed, the methods and products of the present invention thus provide a variety of economic benefits, including reducing of medical costs, decreasing number of days of missed work, and prolonging life, among other benefits.

The present co-inventors have developed a new three-dimensional fiber scaffold, wherein the three-dimensional fiber scaffold is formed with fiber systems comprising materials that have been selected to impart improved strength, flexibility and resorbability characteristics to the scaffold. Therefore, a new generation of drug carriers along with methods of making and using the same have been provided in accordance with the present invention.

### REFERENCES

The publications and other materials listed below and/or set forth in the text above to illuminate the background of the invention, and in particular cases, to provide additional details respecting the practice, are incorporated herein by reference. Materials used herein include but are not limited to the following listed references.

Baker and Kochinke (1989) in “Controlled Release of Drugs: Polymers and Aggregate Systems” pp. 277-306 VCH Publishers, New York.

Langer and Wise, eds (1984) “Medical Applications of Controlled Release” CRC Press, Boca Raton.

Duncan and Seymour (1989) “Controlled Release Technologies: A Survey of Research and Commercial Applications” Elsiver Science Publishers, Oxford, U.K.

Jeong et al. (1999) J Controlled Release 62:109-114.

Kwok et al. (1999) J Controlled Release 62:301-311.

Papahadjopolous, in Ann Rep. Med. Chem. 14: 250-260 (1979) PCT Publication No. WO92/21017 to Unger

U.S. Pat. No. 4,229,447

U.S. Pat. No. 4,769,331

U.S. Pat. No. 5,085,252

U.S. Pat. No. 5,016,652

U.S. Pat. No. 5,279,833

U.S. Pat. No. 5,286,634

U.S. Pat. No. 5,399,346

U.S. Pat. No. 5,465,760

U.S. Pat. No. 5,538,735

U.S. Pat. No. 5,504,086

U.S. Pat. No. 5,646,008

U.S. Pat. No. 5,651,964

U.S. Pat. No. 5,641,484

U.S. Pat. No. 5,643,567

U.S. Pat. No. 5,770,417

U.S. Pat. No. 5,876,452

U.S. Pat. No. 5,980,551

U.S. Pat. No. 6,013,853

It will be understood that various details of the invention can be changed without departing from the scope of the invention. The foregoing description is solely for the purpose of illustration and not for the purpose of limitation, for the invention can admit to other equally effective embodiments.

What is claimed is:

1. A drug carrier, comprising:
(a) a scaffold comprising a plurality of layers and a fiber system interconnecting the plurality of layers, wherein the layers define internal and superficial positions of the scaffold; and
(b) a drug releasably engaged with the scaffold according to a controlled release profile, wherein the drug is releasably engaged at at least one of the internal or superficial positions.

2. A drug carrier, comprising:
(a) a three dimensional fiber scaffold comprising at least three systems of fibers, wherein two of the three fiber systems define the plurality of layers within the three-dimensional fiber scaffold, wherein one of the at least three fiber systems interconnects the plurality of layers, and wherein the three dimensions of the scaffold define internal and superficial positions within the scaffold; and
(b) a drug releasably engaged with the scaffold according to a controlled release profile, wherein the drug is releasably engaged at at least one of the internal or superficial positions.

3. The drug carrier of claim 2, wherein the fiber systems further comprise a monofilament fiber, a multifilament fiber, a hollow fiber, a fiber having a variable cross-section along its length, or combinations thereof.

4. The drug carrier of claim 2, further comprising a plurality of contact points among the at least three fiber systems, and wherein two or more of the at least three fiber systems are secured to each other at one or more of the contact points.

5. The drug carrier of claim 2, wherein the at least three fiber systems in at least one of the plurality of layers define a plurality of interstices within the fiber scaffold.

6. The drug carrier of claim 5, wherein the interstices further comprise a pore size ranging from about 10 μm to about 250 μm.

7. The drug carrier of claim 5, wherein the plurality of layers further comprise an upper layer, a lower layer and a medial layer between the upper layer and the lower layer, and each of the upper, medial, and lower layers have interstices, the medial layer comprising interstices that are smaller than the interstices present in the upper layer, and the lower layer comprising interstices that are smaller than the interstices present in the medial layer.

8. The drug carrier of claim 5, wherein an outer surface of the three-dimensional fiber scaffold is substantially impermeable, thereby restricting drug movement through the outer surface.

9. The drug carrier of claim 1, wherein the drug is impregnated with, coated on, or adhered to the entire scaffold, a single layer of the scaffold, or an individual fiber system of the scaffold; wherein a fiber system comprises the drug; or combinations thereof.

10. The drug carrier of claim 1, wherein the scaffold further comprises a foamed polymer matrix, wherein a plurality of voids are defined by the foamed matrix.

11. The drug carrier of claim 10, wherein the drug is loaded into the voids of the foamed polymer matrix.

12. The drug carrier of claim 1, wherein the drug is engaged with the scaffold such that a concentration of the drug is variable along one or more dimensions of the scaffold.

13. The drug carrier of claim 2, wherein the drug is engaged at internal positions of the scaffold, and wherein the drug is not engaged with fibers residing at superficial positions of the three-dimensional scaffold; or wherein the drug is engaged with fibers residing at superficial positions of the three-dimensional fiber scaffold, and wherein the drug is not engaged with fibers residing at internal positions of the three-dimensional fiber scaffold.

14. The drug carrier of claim 2, wherein the drug is engaged with alternating fiber layers within the scaffold.

15. The drug carrier of claim 14, wherein the plurality of layers further comprise an upper layer, a lower layer and a medial layer, and the upper and lower layers of the fiber scaffold are engaged with a drug, and the medial layer is not engaged with the drug.

16. The drug carrier of claim 1, wherein the carrier is engaged with two or more drugs.

17. The drug carrier of claim 16, wherein each drug is separately engaged with the scaffold.

18. The drug carrier of claim 17, wherein each drug occupies a different or overlapping portion of the scaffold.

19. The drug carrier of claim 18, wherein each drug is released according to a different controlled release profile.

20. The drug carrier of claim 1, wherein the controlled release profile is selected from the group consisting of acute release, continuous release, intermittent release, delayed release, and combinations thereof.

21. The drug carrier of claim 20, wherein the acute release is immediate or delayed.

22. The drug carrier of claim 20, wherein the continuous release further comprises a stable dose over a temporal period of use, an increasing dose over a temporal period of use, or a decreasing dose over a temporal period of use.

23. A method of producing a drug carrier, the method comprising:
(a) forming a scaffold comprising a plurality of layers and a fiber system interconnecting the plurality of layers, wherein the layers define internal and superficial positions of the scaffold; and
(b) releasably engaging a drug with the scaffold at least one of the internal or superficial positions, whereby the drug disengages from the scaffold according to a controlled release profile.

24. A method of producing a drug carrier, the method comprising:
(a) forming a three-dimensional fiber scaffold comprising at least three systems of fibers, wherein two of the three fiber systems define the plurality of layers within the three-dimensional fiber scaffold, wherein one of the at least three fiber systems interconnects the plurality of layers, and wherein the three dimensions of the scaffold define internal and superficial positions within the scaffold; and
(b) releasably engaging a drug with the scaffold at least one of the internal or superficial positions, whereby the drug disengages from the scaffold according to a controlled release profile.

25. The method of claim 24, wherein the fiber systems further comprise a monofilament fiber, a multifilament fiber, a hollow fiber, a fiber having a variable cross-section along its length, or combinations thereof.

26. The method of claim 24, further comprising a plurality of contact points among the at least three fiber systems, and wherein two or more of the at least three fiber systems are secured to each other at one or more of the contact points.

27. The method of claim 23, wherein the at least three fiber systems in at least one of the layers define a plurality of interstices within the scaffold.

28. The method of claim 27, wherein the interstices further comprise a pore size ranging from about 10 μm to about 250 μm.

29. The method of claim 27, wherein the plurality of layers further comprise an upper layer, a medial layer and a lower layer, and the upper, medial, and lower layers have interstices, the medial layer comprising interstices that are smaller than the interstices present in the upper layer, and the lower layer comprising interstices that are smaller than the interstices present in the medial layer.

30. The method of claim 27, wherein the scaffold comprises an outer surface that is substantially free of interstices.

31. The method of claim 30, wherein the outer surface is substantially impermeable, thereby restricting drug movement through the lower layer.

32. The method of claim 23, wherein the drug is impregnated with, coated on, or adhered to the entire scaffold, a single layer of the scaffold, or an individual fiber system of the scaffold; wherein the drug is incorporated within a fiber system during construction of the fiber; wherein a fiber system comprises the drug; or combinations thereof.

33. The method of claim 23, wherein the fiber scaffold further comprises a foamed polymer matrix, wherein a plurality of voids are defined by the foamed matrix

34. The method of claim 33, wherein the drug is loaded into the voids of the foamed polymer matrix.

35. The method of claim 23, wherein the drug is engaged with the scaffold such that a concentration of the drug is variable along one or more dimensions of the scaffold.

36. The method of claim 23, wherein the drug is engaged at internal positions of the scaffold, and wherein the drug is not engaged at superficial positions of the scaffold; or wherein the drug is engaged at superficial positions of the scaffold, and wherein the drug is not engaged at internal positions of the scaffold.

37. The method of claim 23, wherein the drug is engaged with alternating layers within the scaffold.

38. The method of claim 23, wherein the plurality of layers comprises an upper layer, a medial layer and a lower layer, and the upper and lower layers of the scaffold are engaged with a drug, and the medial layer is not engaged with the drug.

39. The method of claim 23, wherein the carrier is engaged with two or more drugs.

40. The method of claim 39, wherein each drug is separately engaged with the scaffold.

41. The method of claim 39, wherein each drug occupies a different or overlapping portion of the scaffold.

42. The method of claim 40, wherein each drug is releasably attached according to a different controlled release profile.

43. The method of claim 23, wherein the controlled release profile is selected from the group consisting of acute release, continuous release, intermittent release, delayed release, continuously increasing release, continuously decreasing release and combinations thereof.

44. The method of claim 43, wherein the acute release is immediate or delayed.

45. The method of claim 43, wherein the continuous release further comprises a stable dose over a temporal period of use, an increasing dose over a temporal period of use, or a decreasing dose over a temporal period of use.

46. A method of delivering a drug to a subject, the method comprising:
(a) providing a drug carrier comprising:
(i) a scaffold comprising a plurality of layers and a fiber system interconnecting the plurality of layers, wherein the layers define internal and superficial positions of the scaffold; and
(ii) a drug releasably engaged with the scaffold according to a controlled release profile, wherein the drug is releasably engaged at at least one of the internal or superficial positions; and
(b) placing the carrier at a site of desired exposure to the drug whereby disengagement of the drug from the scaffold occurs according to the controlled release profile.

47. A method of delivering a drug to a subject, the method comprising:
(a) providing a drug carrier comprising:
(i) a three dimensional fiber scaffold of at least three systems of fibers, wherein two of the three fiber systems define the plurality of layers within the three-dimensional fiber scaffold, wherein one of the at least three fiber systems interconnects the plurality of layers, and wherein the three dimensions of the scaffold define internal and superficial positions within the scaffold; and
(ii) a drug releasably engaged with the scaffold according to a controlled release profile, wherein the drug is releasably engaged at at least one of the internal or superficial positions; and
(b) placing the carrier at a site of desired exposure to the drug whereby disengagement of the drug from the scaffold occurs according to the controlled release profile.

48. The method of claim 47, wherein the fiber systems further comprise a monofilament fiber, a multifilament fiber, a hollow fiber, a fiber having a variable cross-section along its length, or combinations thereof.

49. The method of claim 47, further comprising a plurality of contact points among the at least three fiber systems, and wherein two or more of the at least three fiber systems are secured to each other at one or more of the contact points.

50. The method of claim 47, wherein the at least three fiber systems in at least one of the layers define a plurality of interstices within the fiber scaffold.

51. The method of claim 50, wherein the interstices further comprise a pore size ranging from about 10 μm to about 250 μm.

52. The method of claim 50, wherein the plurality of layers further comprises an upper layer, a medial layer and a lower layer, and each of the upper, medial, and lower layers have interstices, the medial layer comprising interstices that are smaller than the interstices present in the upper layer, and the lower layer comprising interstices that are smaller than the interstices present in the medial layer.

53. The method of claim 46, wherein the scaffold comprises an outer surface have at least a portion that is free of interstices.

54. The method of claim 53, wherein the portion of the outer surface is substantially impermeable, thereby prohibiting drug movement through the outer surface.

55. The method of claim 46, wherein the drug is impregnated with, coated on, or adhered to the entire scaffold, a single layer of the scaffold, or an individual fiber system of the scaffold; wherein the drug is loaded into a hollow fiber through an open end of the hollow fiber; wherein the drug is incorporated within a fiber system during construction of the fiber; wherein the fiber system comprises the drug; or combinations thereof.

56. The method of claim 46, wherein the scaffold further comprises a foamed polymer matrix, wherein a plurality of voids are defined by the foamed matrix.

57. The method of claim 56, wherein the drug is loaded into the voids of the foamed polymer matrix.

58. The method of claim 46, wherein the drug is engaged with the scaffold such that a concentration of the drug is variable along one or more dimensions of the scaffold.

59. The method of claim 46, wherein the drug is engaged with fibers residing at internal positions of the scaffold, and wherein the drug is not engaged with fibers residing at superficial positions of the scaffold; or wherein the drug is engaged at superficial positions of the scaffold, and wherein the drug is not engaged at internal positions of the scaffold.

60. The method of claim 46, wherein the drug is engaged with alternating layers within the scaffold.

61. The method of claim 60, wherein the plurality of layers further comprises an upper layer, a lower layer and a medial layer, and the upper and lower layers of the fiber scaffold are engaged with a drug, and the medial layer is not engaged with the drug.

62. The method of claim 46, wherein the carrier is engaged with two or more drugs.

63. The method of claim 62, wherein each drug is separately engaged with the scaffold.

64. The method of claim 63, wherein each drug occupies a different or overlapping portion of the scaffold.

65. The method of claim 64, wherein each drug is released according to a different controlled release profile.

66. The method of claim 46, wherein the controlled release profile is selected from the group consisting of acute release, continuous release, intermittent release, delayed release, continuously increasing release, continuously decreasing release and combinations thereof.

67. The method of claim 66, wherein the acute release is immediate or delayed.

68. The method of claim 67, wherein the continuous release further comprises a stable dose over a temporal period of use, an increasing dose over a temporal period of use, or a decreasing dose over a temporal period of use.

69. The method of claim 46, wherein the desired site of drug release is an internal body site or an external body site.

70. The method of claim 69, further comprising implanting the carrier within a subject at the internal site.

71. The method of claim 69, further comprising adhering the carrier to or wrapping the carrier around the external body site.
